(19) United States
(12) Patent Application Publication
(10) Pub. No.: US 2023/0241044 A1
(43) Pub. Date: Aug. 3, 2023

(54) TREATMENT FOR PSORIASIS AND SKIN INFLAMMATORY DISEASES

(71) Applicant: ZYDUS LIFESCIENCES LIMITED

(72) Inventors: Mukul JAIN, Ahmedabad, Gujarat (IN); Amit JOHARAPURKAR, Ahmedabad, Gujarat (IN); Vishal PATEL, Ahmedabad, Gujarat (IN)

(21) Appl. No.: 17/928,557

(22) PCT Filed: May 28, 2021
(86) PCT No.: PCT/IB2021/054685

§ 371 Date: Nov. 29, 2022

(30) Foreign Application Priority Data
May 29, 2020 (IN) 202021022542

(51) Int. Cl. A61K 31/4418; A61K 31/5377; A61K 45/06; A61K 31/515; A61K 31/4704; A61P 17/06

(52) U.S. Cl. CPC A61K 31/4418; A61K 31/515; A61K 31/4704; A61K 31/5377; A61K 45/06; A61P 17/06

(57) ABSTRACT

Present invention relates to development of prolyl hydroxylase inhibitor for treatment of Psoriasis and skin inflammatory diseases. Specifically, invention relates to development of compound of formula (Ia) for treatment of Psoriasis and skin inflammatory diseases. Invention also relates to pharmaceutical composition comprising compound of formula (Ia) for treatment of Psoriasis and skin inflammatory diseases.

### FIELD OF THE INVENTION

The present invention is related to the development of therapeutic compound for the treatment of Psoriasis and skin inflammatory diseases. The present invention further provides a prolyl hydroxylase inhibitor or its pharmaceutically acceptable salt or suitable composition useful in the treatment of Psoriasis and skin inflammatory diseases. Specifically the invention relates to development of compound of formula (Ia) for the treatment of Psoriasis and skin inflammatory diseases. Further, the invention also relates to pharmaceutical composition of compound of formula (Ia) for the treatment of Psoriasis and skin inflammatory diseases.

### BACKGROUND OF THE INVENTION

Psoriasis is a chronic inflammatory condition of skin characterized by epidermal hyperplasia, and infiltration of monocytes, neutrophil, dendritic cells, and T lymphocytes. Psoriasis is associated with papules and scales, due to an abnormal homeostasis between epidermis and immune system which leads to premature maturation of keratinocytes and inflammatory cell infiltration in the dermis and abnormal hyper proliferation of the skin’s epidermal layer (Griffiths et al, 2007). Pathogenesis of psoriasis involves both Th1 and Th17 cells. The inflammatory cytokines involved are IFN-γ, IL-2, IL-18, IL-17A, IL-17F, IL-22, IL-26, and TNF-α are increased in serum and skin (Tesmer et al., 2008). Current therapy for psoriasis is limited to treatment of inflammatory episodes with anti-inflammatory agents such as corticosteroids, apremilast, or biologicals agents that inhibit action of cytokine (Turbeville et al., 2017). Most common clinically evident psoriasis is chronic plaque or psoriasis vulgaris. Psoriasis is associated with much comorbidity, of which psoriatic arthritis is most common (Helliwell et al, Ann Rheum Dis 2005).

Many pharmacological therapies for psoriasis have been explored. These include topical treatments like vitamin D and its analogues, corticosteroids, calcineurin inhibitors, dithranol, and tar, phototreatments, and oral or injectable therapies that involve methotrexate, ciclosporin, retinoid, and fumarates. Biological agents that target tumor necrosis factors α, interleukin 12, or interleukin 23, are also been developed (Griffiths et al, Lancet 2007; 370: 263-71). However, the long-term safe control of psoriasis is limited due to variations in individual responsiveness, comorbidities, and side effects of the existing therapies. In addition, toxicity and patient compliance of the injectable therapies (which include biological agents) remains an issue in the long term therapeutic success.

Oxygen is an important factor which regulates acute and chronic inflammation. Oxygen levels in the tissues are sensed by hypoxia-inducible factors (HIFs: HIF-1 and HIF-2), and HIF is widely expressed in inflammatory cells such as neutrophils, lymphocytes and other TH17 cells (Dang et al., 2011; McNamee et al., 2013; Shi et al., 2011; Walmsley et al., 2005). LPS-induced Inflammation stimulates HIF, and TNF-α and NF-kB also induces HIF expression (Albina et al., 2001; Blouin et al., 2004; Fitzpatrick et al., 2011). HIF has been reported to regulate nuclear factor-κB (NF-κB) and extracellular signal-regulated kinase (ERK) mediated inflammatory and fibrotic pathways (Scholz et al., 2013). The hypoxia-inducible factor (HIF) is a major regulator of immune cell survival and function in skin. Prolyl hydroxylase (PHD) enzymes cause hydroxylation of HIF and thus direct HIF towards degradation. Inhibition of PHD can stabilize HIF thus increasing the availability of HIF at the site of inflammation. PHD also regulates fibrotic process in the body (Robinson et al., 2008; Tambuwala et al., 2010). Recently, it is reported that HIF-PHD inhibition prevents inflammation in keratinocytes and ameliorated allergic contact dermatitis (Manresa et al., 2019; Tashiro et al., 2019). Using a PHD inhibitor HIF can be stabilized and thus skin inflammation be regulated. It is also reported that HIF regulates inflammation in Th1 and Th17 cells (Marks et al., 2017). Compound of formula (Ia) is a PHD inhibitor currently in phase 3 clinical trials. It is reported that compound of formula (Ia) treatment stabilizes HIF and thus induces erythropoiesis in animal model of anemia (Jain et al., 2019; Joharapurkar et al., 2018). Compound of formula (Ia) also improves haemoglobin in clinical trials.

Some of the prolyl hydroxylase inhibitors have been disclosed in EP661269, WO2007070359, WO2008076425, WO2011007856, WO2012106472, and WO2013043621. Specifically WO2004108681 and WO2008002576 covers the prolyl hydroxylase inhibitors named Roxadustat and Vadadustat respectively.

WO2014102818 discloses compounds of the following general formula

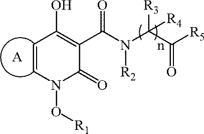

These compounds are reported to be useful for the treatment of anemia. It has surprisingly now been found that compound of formula (Ia) as given below:

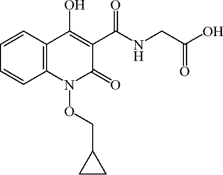 [SMILES: O=C(O)CNC(=O)c1c(O)c2ccccc2n(OCC2CC2)c1=O]

and its pharmaceutically acceptable salts or pharmaceutical compositions are effective in the treatment of psoriasis and skin inflammatory diseases also.

### EMBODIMENTS OF THE INVENTION

In an embodiment the present invention provides prolyl hydroxylase inhibitors suitable for the treatment of Psoriasis and skin inflammatory diseases

In another embodiment the present invention provides a suitable composition comprising prolyl hydroxylase inhibitors and suitable pharmaceutically acceptable excipients for the treatment of Psoriasis and skin inflammatory diseases.

In an embodiment the present invention provides a compound of formula (Ia) and their pharmaceutically acceptable salts suitable for the treatment of Psoriasis and skin inflammatory diseases.

In another embodiment the present invention provides a suitable composition comprising the compound of formula (Ia) and suitable pharmaceutically acceptable excipients for the treatment of Psoriasis and skin inflammatory diseases.

In yet another embodiment the present invention provides the administration of compound of formula (Ia) and their pharmaceutically acceptable salts alone or in combination with other suitable agents as therapeutic agent for the treatment of Psoriasis and skin inflammatory diseases.

### SUMMARY OF THE INVENTION

The present invention provides a prolyl hydroxylase inhibitors suitable for the treatment of Psoriasis and skin inflammatory diseases. More specifically the present invention provides a compound of formula (Ia) and their pharmaceutically acceptable salts for the treatment of psoriasis and skin inflammatory diseases. Moreover, the present invention also provides a suitable composition comprising compound of formula (Ia) and their pharmaceutically acceptable salts and suitable pharmaceutically acceptable excipients useful in the treatment of psoriasis and skin inflammatory diseases.

### DESCRIPTION OF ACCOMPANYING FIGURES

FIG. 1. The effect of topical application of compound of formula (Ia) on IMQ-induced psoriasis-like skin lesions in mice.

FIG. 2. The effect of topical application of compound of formula (Ia) on ear thickness and skinfold thickness in IMQ-induced psoriasis in mice.

FIG. 3. The effect of topical application of compound of formula (Ia) on ear thickness in Mannan-induced psoriasis in mice.

### DESCRIPTION OF THE INVENTION

The term ‘treating’ or ‘treatment’ or condition as used herein means: preventing or delaying the appearance of clinical symtomps of the state, disorder or condition developing in a mammal.

The term ‘preventing’ refers to barring a subject from acquiring a disorder or disease in the first place.

The term ‘pharmaceutically acceptable’ use embraces both human and veterinary use.

The present invention describes prolyl hydroxylase inhibitors suitable for the treatment of Psoriasis and skin inflammatory diseases.

Wherein prolyl hydroxylase inhibitors are selected from Roxadustat, Molidustat, Desidustat (compound of formula (Ia)) and Daprodustat.

In an embodiment is provided suitable pharmaceutical composition for the treatment of psoriasis and skin inflammatory diseases comprising the compound of Roxadustat, Molidustat and Daprodustat or its pharmaceutically acceptable salt along with suitable excipients as defined hereinafter.

In another embodiment, the present invention provides a method of treating a subject suffering from psoriasis and skin inflammatory diseases which comprises treatment of a patient in need of such therapy, with Roxadustat, Molidustat and Daprodustat or its pharmaceutically acceptable salt or suitable pharmaceutical compositions containing them.

In a further embodiment the present invention provides use of the Roxadustat, Molidustat and Daprodustat or its pharmaceutically acceptable salt or their suitable pharmaceutical compositions for the treatment of psoriasis and skin inflammatory diseases.

The effective amount of the Roxadustat, Molidustat and Daprodustat is selected from 1 mg to 500 mg preferably 1 mg to 250 mg and more preferably 4 mg to 50 mg.

In one embodiment the present invention disclose Roxadustat, Molidustat and Daprodustat or its pharmaceutically acceptable salt or suitable pharmaceutical compositions can also be used for the treatment of other skin inflammatory diseases such as: dermatomycosis, scleroderma, epidermolysis bullosa, eczema and systemic lupus erythematous affecting skin. It can also be used for the treatment of hives, acneiform eruptions, auto inflammatory diseases (Blau syndrome, Majeed syndrome, Muckle-Wells syndrome), chronic blistering diseases, skin mucus diseases, inflammation of skin appendages, diseases of alteration in pigmentation, drug-induced skin diseases, eosinophilic cutaneous conditions, bacterial or viral or fungal or parasite skin infections, lichen planus, lymphoid-related cutaneous conditions, monocyte-and macrophage-related cutaneous inflammation, reactive neutrophilic cutaneous condition, utricaria and other skin inflammation of unknown origin.

In an embodiment the present invention provides Roxadustat, Molidustat and Daprodustat or its pharmaceutically acceptable salts is administrated orally, intravenously, parentally or topically in the subject who is in need of treatment.

In yet another embodiment the present invention provides the administration of Roxadustat, Molidustat and Daprodustat and their pharmaceutically acceptable salts alone or in combination with other suitable agents as therapeutic agent for the treatment of Psoriasis and skin inflammatory diseases.

In an embodiment, the additional therapeutic agent used is selected from a PDE4 inhibitor, methotrexate or derivatives thereof, cyclosporine or derivatives thereof, vitamins D, E and A or derivatives thereof, glucocorticoid or derivatives thereof, anti-inflammatory agents, immunosuppressive agents, antihistamines, monoclonal antibodies and fusion proteins, cytokines and mediators, antibiotic or derivatives thereof, antifungal agents or derivatives thereof, hormones, peroxisome proliferator-activated receptor gamma activators or derivatives thereof, anti-viral agents or derivatives thereof, vegetable and/or animal extracts, phytochemicals or derivatives thereof, zinc pyrithione and/or other essential minerals, phototherapy and cell hyper proliferation modulators.

In a Preferred embodiment the present invention provides compound of formula (Ia) which is suitable for the treatment of Psoriasis and skin inflammatory diseases.

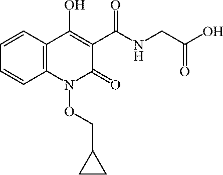 [SMILES: O=C(O)CNC(=O)c1c(O)c2ccccc2n(OCC2CC2)c1=O]

In an embodiment is provided suitable pharmaceutical composition for the treatment of psoriasis and skin inflammatory diseases comprising the compound of formula (Ia) or its pharmaceutically acceptable salt along with suitable excipients as defined hereinafter.

In another embodiment, the present invention provides a method of treating a subject suffering from psoriasis and skin inflammatory diseases which comprises treatment of a patient in need of such therapy, with compound of formula (Ia) or its pharmaceutically acceptable salt or suitable pharmaceutical compositions containing them.

The pharmaceutical acceptable salts of the compound of formula (Ia) is inorganic metal salt or organic amines salts.

Wherein inorganic metal salt is selected from calcium, sodium, potassium, lithium, barium, strontium, magnesium, cesium, copper, cobalt, iron, manganese, lead, aluminium, cadmium, silver, zinc, ammonium and the like;

Wherein organic amines salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (-)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane1-6-diamine, 2-(2-aminoethoxy)ethanamine, N-methylmorpholine, and N-ethylmorpholine.

In an embodiment, the effective amount of the said compound of formula (Ia) is selected from 25 mg to 250 mg preferably 50 mg to 150 mg.

In other preferred embodiment, the pharmaceutical composition comprising effective amount of compound of formula (Ia) is ranging from 1% to 10%w/w.

In one of the embodiment a compound of formula (Ia) or its pharmaceutically acceptable salt or suitable pharmaceutical compositions can also be used for the treatment of other skin inflammatory diseases such as: dermatomycosis, scleroderma, epidermolysis bullosa, eczema and systemic lupus erythematous affecting skin. It can also be used for the treatment of hives, acneiform eruptions, autoinflammatory diseases (Blau syndrome, Majeed syndrome, Muckle-Wells syndrome), chronic blistering diseases, skin mucus diseases, inflammation of skin appendages, diseases of alteration in pigmentation, drug-induced skin diseases, eosinophilic cutaneous conditions, bacterial or viral or fungal or parasite skin infections, lichen planus, lymphoid-related cutaneous conditions, monocyte-and macrophage-related cutaneous inflammation, reactive neutrophilic cutaneous condition, utricaria and other skin inflammation of unknown origin.

In an embodiment the present invention provides a compound of formula (Ia) or its pharmaceutically acceptable salts is administrated orally, intravenously, parentally or topically in the subject who is in need of treatment. In a preferred embodiment the present invention provides a compound of formula (Ia) or its pharmaceutically acceptable salt is to be administered orally or topically.

In yet another embodiment the present invention provides the administration of compound of formula (Ia) and their pharmaceutically acceptable salts alone or in combination with other suitable agents as therapeutic agent for the treatment of Psoriasis and skin inflammatory diseases.

In an embodiment, the additional therapeutic agent used is selected from a PDE4 inhibitor, methotrexate or derivatives thereof, cyclosporine or derivatives thereof, vitamins D, E and A or derivatives thereof, glucocorticoid or derivatives thereof, anti-inflammatory agents, immunosuppressive agents, antihistamines, monoclonal antibodies and fusion proteins, cytokines and mediators, antibiotic or derivatives thereof, antifungal agents or derivatives thereof, hormones, peroxisome proliferator-activated receptor gamma activators or derivatives thereof, anti-viral agents or derivatives thereof, vegetable and/or animal extracts, phytochemicals or derivatives thereof, zinc pyrithione and/or other essential minerals, phototherapy and cell hyper proliferation modulators.

In an embodiment the present invention also provides a suitable pharmaceutical composition of compounds of formula (Ia) or its pharmaceutically acceptable salts. The pharmaceutical composition of the present invention essentially comprises of:
the pharmaceutically active substance of formula (Ia) its pharmaceutically acceptable salts;
optionally with one or more pharmaceutically acceptable excipients.

The pharmaceutically acceptable excipients are selected at least one from diluent, binders, disintegrating agents, lubricating agents, glidant agent, coating redimix, gelling agent, humectant, chelating agents, permeation enhancers, preservatives, antioxidants, solubilizing agents, acidifying/alkalizing agent, Emollient, Emulsifying agents and the like

Diluents include, but are not limited to starch and its processed and co-processed derivertives, saccharides, di saccharides, sucrose, lactose, polysaccharides, cellulose, cellulose ethers, cellulose acetate, hydroxypropyl cellulose, sugar alcohols, xylitol, sorbitol, maltitol, lactitol, microcrystalline cellulose, magnesium or calcium or sodium carbonate, lactose, lactose monohydrate, di-calcium phosphate, compressible sugars, di-basic calcium phosphate dihydrate, mannitol lactose anyhydrous, magnesium oxide, maltodextrin, maltose, pullulan, sodium alginate, sodium bicarbonate, calcium silicate, calcium sulphate, cell and tribasic calcium phosphate or suitable combinations thereof and other such materials known to those of ordinary skill in the art.

Binders include, but are not limited to hypromellose 3 Cps, carbomers selected from carbopol, gellan, gum Arabic, hydrogenated vegetable oil, polymethacrylates selected from Eudragit, xanthan, lactose and Zein combinations thereof and other such materials known to those of ordinary skill in the art.

Disintegrating agents include, maize starch, sodium starch glycolate, croscarmellose sodium, crospovidone, microcrystalline cellulose, modified com starch, sodium carboxymethyl starch, povidone, pregelatinized starch, agar, carboxymethyl cellulose calcium or sodium, colloidal silicon dioxide, chitosan, docusate sodium, hydroxyl propyl cellulose, magnesium aluminium silicate, maltose, methyl cellulose, polacrilin potassium, and alginic acid or suitable combinations thereof and other such materials known to those of ordinary skill in the art.

Glidant agents include, but are not limited to, colloidal silica, calcium silicate, magnesium silicate, silicon hydrogel, cornstarch, talc, combinations thereof and other such materials known to those of ordinary skill in the art.

Lubricating agents include, but are not limited to, magnesium stearate, stearic acid, silica, fats, zinc or sucrose or sodium or calcium stearate, castor oil, hydrogenated castor oil, . Polyethylene glycol and its derivatives, sodium stearyl fumarate, talc, or fatty acids including lauric acid, oleic acid, glyceryl behenate, glyceryl monostearate, and C₁-C₁₀ fatty acid or suitable combinations thereof and other such materials known to those of ordinary skill in the art.

Coating redimix is selected from Opadry Pink all such materials known to those of ordinary skill in the art.

Solubilizers/co-solvents used anywhere in the description may be selected from Dimethyl malonate, diethyl succinate, diethyl glutarate, diethyl adipate, dipropyl adipate, dibutyl sebacate, diisopropyl sebacate, diethyl pimelate, diethyl suberate, diethyl azelate, dibutyl adipate, dibutyl sebacate, methyl ethyl succinate, diethyl ethyl-isopropylmalonate, diethyl isosuccinate, benzyl alcohol, benzyl benzoate, cyclodextrin, glycerine monostearate, lecithin, butylene glycol, dibutyl phthalate, diethyl phthalate, dimethyl ether, diethyl ether, ethyl acetate, ethyl lactate, ethyl oleate, glycofurol, isopropyl alcohol, triacetin, triethanolamine, hexylene glycol, dimethyl sulfoxide (DMSO) and/or dimethyl isosorbide, propylene glycol, glycerin, Diethylene glycol monoethyl ether, dimethyl acetamide, polyethylene glycol, polysorbate 80, 60 & 20, purified water, ethanol and suitable mixture thereof.

Permeation enhancers used anywhere in the description may be selected from polyethylene glycol, polyethylene glycol monolaurate, butanediol, dimethylsulfoxide, decylmethylsulfoxide, diethylene glycol monoethyl ether (e.g., Transcutol® P), lauric acid, oleic acid, valeric acid, isopropyl myristate, isopropyl palmitate, methyl propionate, and ethyl oleate; urea, dimethyl acetamide, dimethylformamide 2- pyrrolidone, ethanolamine, methyl-2 -pyrrolidone, diethanolamine, triethanolamine, terpenes, alkanones, salicylic acid, citric acid, succinic acid and suitable mixtures thereof.

Humectants used anywhere in the description may be selected from glycerin, propylene glycol, Polyethylene glycol, sorbitol solution, 1,2,6 -hexanetriol and suitable mixtures thereof.

Antioxidants used anywhere in the description may be selected from ascorbic acid (vitamin C), glutathione, lipoic acid, uric acid, carotenes, a-tocopherol (vitamin E), ubiquinol, butylated hydroxyanisole, butylated hydroxytoluene, sodium benzoate, sodium thiosulphate, sodium metabisulphite, propyl gallate (PG, E310), and tertiary-butylhydroquinone, Idebenone, Lycopene and suitable mixtures thereof.

Preservatives used anywhere in the description may be selected from Methyl paraben, Propyl paraben, benzoic acid, imidurea, sorbic acid, potassium sorbate, benzalkonium chloride, phenyl mercuric acetate, chlorobutanol, phenoxyethanol, benzyl alcohol, chlorocresol, metacresol, cetrimonium chloride, benzethonium chloride, sodium edetate, boric acid, phenol and suitable mixtures thereof.

Chelating agents used anywhere in the description may be selected from EDTA, disodium EDTA, trisodium EDTA, EGTA, disodium EGTA, trisodium EGTA, citric acid, phosphoric acid, succinic acid, and suitable mixtures thereof.

Acidifying/alkalizing agents used anywhere in the description may be selected from trimethylamine, triethylamine, pyridine, picoline, ethanolamine, diethanolamine, triethanolamine, meglumine, dicyclohexylamine, N,N′-dibenzylethylenediamine, arginine, lysine, ornithine, sodium bicarbonates, sodium hydroxide, potassium hydroxide and suitable mixtures thereof.

Buffers used anywhere in the description may be selected from citrate/citric acid buffers, acetate/acetic acid buffers, phosphate/phosphoric acid buffers, formate/formic acid buffers, propionate/propionic acid buffers, carbonate/carbonic acid buffers, ammonium/ammonia buffers and suitable mixtures thereof.

Gelling agents used anywhere in the description may be selected from carbomer, Methyl cellulose, sodium carboxy methyl clellulose, Carrageenan, colloidal silicon dioxide, Guar gum, hydroxypropyl methyl cellulose, hydroxyl ethyl cellulose, hydroxy propyl cellulose, Gelatin, polyethyene oxide, alginic acid, sodium alginate, fumed silica, polyvinylpyrrolidone, polyvinyl alcohol and suitable mixtures thereof.

Emollient/ stiffening agents used anywhere in the description may be selected from carnauba wax, cetyl alcohol, cetyl ester wax, hydrous lanolin, lanolin, lanolin alcohols, paraffin, petrolatum. polyethylene glycol, stearic acid, stearyl alcohol, white wax, yellow wax, liquid paraffin, liquid petrolatum, jojoba oil, sesame oil, rapeseed oil, purcellin oil, 2-ethylhexyl palmitate, 2-octyldodecyl stearate, 2-octyldodecyl erucate, isostearyl isostearate, 2-octyldodecyl benzoate, triglycerides of caprylic/capric acids, octyldodecanol, isohexadecane, capmul MCM and suitable mixtures thereof.

Emulsifying agents used anywhere in the description may be selected from polysorbate 20, polysorbate 60, polysorbate 80, poloxamer, emulsifying wax, sorbitan monostearate, sorbitan monooleate, sodium lauryl sulphate, propylene glycol monostearate, glyceryl monostearate and suitable mixtures thereof.

Ointment bases used anywhere in the description may be selected from oleaginous bases such as petrolatum, white/yellow petrolatum, liquid paraffin, hard paraffin, white ointment; absorption bases such as lanolin, anhydrous lanolin, cold cream, etc.; water removable bases: hydrophilic ointments, vanishing creams and water; water soluble bases such as polyethylene glycol 200, 300, 400, 1500, 3000, 6000 and suitable mixtures thereof.

The pharmaceutical composition comprising effective amount of the said compound of formula (Ia) is selected from 25 mg to 250 mg preferably 50 mg to 150 mg.

In one of the preferred embodiment, the present pharmaceutical composition comprising effective amount of compound of formula (Ia) is ranging from 0.1% to 25% w/w.

In one of the embodiment pharmaceutical compositions can also be used for the treatment of other skin inflammatory diseases such as: dermatomycosis, scleroderma, epidermolysis bullosa, eczema and systemic lupus erythematous affecting skin. It can also be used for the treatment of hives, acneiform eruptions, autoinflammatory diseases (Blau syndrome, Majeed syndrome, Muckle-Wells syndrome), chronic blistering diseases, skin mucus diseases, inflammation of skin appendages, diseases of alteration in pigmentation, drug-induced skin diseases, eosinophilic cutaneous conditions, bacterial or viral or fungal or parasite skin infections, lichen planus, lymphoid-related cutaneous conditions, monocyte-and macrophage-related cutaneous inflammation, reactive neutrophilic cutaneous condition, utricaria and other skin inflammation of unknown origin.

In an embodiment the present invention provides a pharmaceutical composition comprising compound of formula (Ia) or its pharmaceutically acceptable salts is administrated orally, intravenously, parentally or topically in the subject who is in need of treatment.

In a preferred embodiment the present invention provides a pharmaceutical composition comprising compound of formula (Ia) or its pharmaceutically acceptable salt is to be administered orally or topically.

In an embodiment the present invention provides the administration of pharmaceutical composition of compound of formula (Ia) further comprising additional therapeutic agents for the treatment of Psoriasis and skin inflammatory diseases.

In another embodiment, the additional therapeutic agent used in is selected from a PDE4 inhibitor, methotrexate or derivatives thereof, cyclosporine or derivatives thereof, vitamins D, E and A or derivatives thereof, glucocorticoid or derivatives thereof, anti-inflammatory agents, immunosuppressive agents, antihistamines, monoclonal antibodies and fusion proteins, cytokines and mediators, antibiotic or derivatives thereof, antifungal agents or derivatives thereof, hormones, peroxisome proliferator-activated receptor gamma activators or derivatives thereof, anti-viral agents or derivatives thereof, vegetable and/or animal extracts, phytochemicals or derivatives thereof, zinc pyrithione and/or other essential minerals, phototherapy and cell hyper proliferation modulators.

In yet another embodiment the present invention provides method of treating Psoriasis and skin inflammatory diseases using compound of formula (Ia) or its pharmaceutically acceptable salts.

The compound of formula (Ia) is known as Desidustat. The compound of formula (Ia), Roxadustat, Vadadustat and Daprodustat may be prepared by any of the methods known in the art including those processes disclosed in the prior art such as those mentioned elsewhere in the specification.

While the present invention has been described in terms of a few specific aspects, modifications and equivalents thereof, in light of teaching and disclosure of the present invention, which are apparent of the skilled artisan, are to be construed as included within the scope of the invention.

### Example 1

The efficacy of the compound in the treatment of psoriasis is evaluated as follows:

### Imiquimod-Induced Psoriasis

### Study Design and Treatment

The 8 to 10 week old male C57 mice (18-20 g) received a daily topical dose of commercially available Imiquimod (IMQ) cream (5%, Imiquad;) on the shaved back and the right ear for 5 consecutive days, translating in a daily dose of 3.125 mg of the active compound to establish a model of IMQ-induced psoriasis. To examine the efficacy of topical solution of compound of formula (Ia), mice were randomly divided into 3 groups: IMQ-induced model group (IMQ) and IMQ + 5% compound of formula (Ia) solution and control group. Treatment was started on day 1 after the topical application of IMQ and continued upto day 5. All animals were assessed for the severity of the psoriasis-like skin condition, using 3 elements of the Psoriasis Area Severity Index (PASI), to assign a score of 0-4 (0, none; 1, mild; 2, moderate; 3, severe; 4, very severe) for each of the parameters erythema, scaling and thickness. The cumulative score (erythema plus scaling plus thickness) served to indicate the severity of inflammation (scale 0-12). Ear thickness was measured every day using thickness meter.

### Results

### Compound of Formula (Ia) Treatment Reduces IMQ-Induced Skin Inflammation and Ear Thickness in Mice

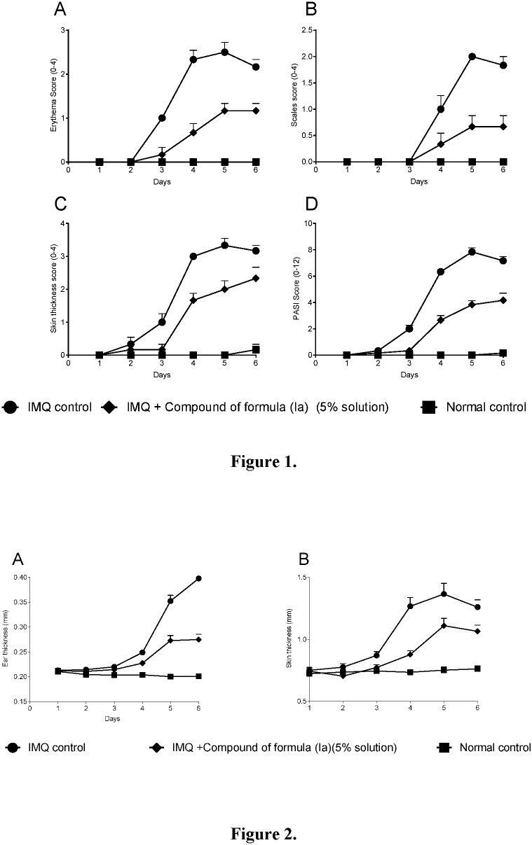

The psoriasis-like skin conditions became apparent from day 3 onwards in IMQ group (FIG. 1). Erythema score and skin thickness score started to appear from day 3 while scale score was apparent from day 4 of the IMQ treatment (FIGS. 1A-C). IMQ application increased PASI score from day onwards (FIG. 1D). Topical application of compound of formula (Ia) reduced erythema score 46.2 ± 7.7% erythema score by 63.6 ± 11.5% and skin thickness score by 26.3 ± 10.4% when compared with IMQ-control (FIGS. 1A-C). Compound of formula (Ia) decreased PASI score to 41.9 ± 7.6 % when compared with IMQ-control (FIG. 1D). Thickness of the right ears the IMQ group was increased to 0.40 mm from 0.21 mm (FIG. 2A). In the compound of formula (Ia) treatment group, thickening of the ear was significantly reduced to 30.8 ± 2.6% compared to the IMQ-control (FIG. 2A). Back skinfold thickness of the mice in the IMQ-control increased from 0.75 mm to 1.26 mm (FIG. 2B). Topical application of compound of formula (Ia) reduced skinfold thickness by 15.5 ± 4.0%, compared to the IMQ (FIG. 2B).

### Example 2

### Mannan-Induced Psoriasis in Mice

### Study Design and Treatment

The 8- to 10-week-old male C57 mice (18-20 g) received a single dose of mannan from Saccharomyces cerevisiae (20 mg/kg, intraperitoneal route, dissolved in saline). To examine the efficacy of topical solution of desidustat, mice were randomly divided into 3 groups: Normal control, mannan control- model group (MAN control), and mannan (MAN) + 2.5% compound of formula (Ia) solution. Normal control animals were given normal saline instead of mannan. Treatment was started on day 0 along with injection of mannan and continued up to day 5. Right ear thickness was measured every day using thickness meter.

### Results

Compound of formula (Ia) treatment reduces Mannan-induced ear thickness in mice

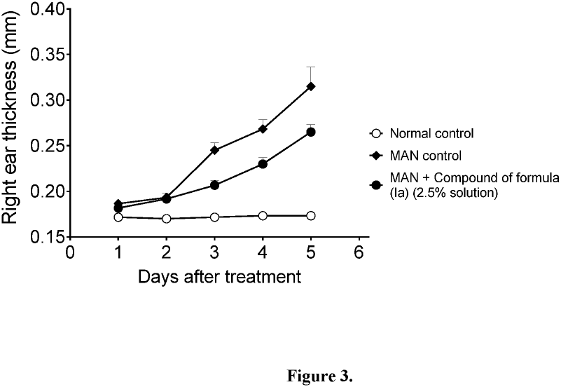

The right ear thickness was appeared to increase from day 2 to day 5 in MAN control group (FIG. 3). Treatment with 2.5% Compound of formula (Ia) reduced right ear thickness by 15.8 ± 2.5%, on day 5, when compared to MAN control group.

### REFERENCES

Griffiths C.E.M., Barker J.N.W.N. 2007. Pathogenesis and clinical features of psoriasis. Lancet. 370: 263-71.

Tesmer, L.A., Lundy, S.K., Sarkar, S., Fox, D.A., 2008. Th17 cells in human disease. Immunol. Rev. 223, 87-113.

Turbeville, J.G., Patel, N.U., Cardwell, L.A., Oussedik, E., Feldman, S.R., 2017. Recent Advances in Small Molecule and Biological Therapeutic Approaches in the Treatment of Psoriasis. Clin. Pharmacol. Ther. 102, 70-85.

Helliwell P.S., Taylor W.J. 2005. Classification and diagnostic criteria for psoriatic arthritis. Ann Rheum Dis 2005; 64: ii3-ii8.

Dang, E. V., Barbi, J., Yang, H.Y., Jinasena, D., Yu, H., Zheng, Y., Bordman, Z., Fu, J., Kim, Y., Yen, H.R., Luo, W., Zeller, K., Shimoda, L., Topalian, S.L., Semenza, G.L., Dang, C. V., Pardoll, D.M., Pan, F., 2011. Control of TH17/Treg balance by hypoxia-inducible factor 1. Cell 146, 772-784.

McNamee, E.N., Korns Johnson, D., Homann, D., Clambey, E.T., 2013. Hypoxia and hypoxia-inducible factors as regulators of T cell development, differentiation, and function. Immunol. Res. 55, 58-70

Shi, L.Z., Wang, R., Huang, G., Vogel, P., Neale, G., Green, D.R., Chi, H., 2011. HIF1α-dependent glycolytic pathway orchestrates a metabolic checkpoint for the differentiation of TH17 and Treg cells. J. Exp. Med. 208, 1367-1376.

Walmsley, S.R., Print, C., Farahi, N., Peyssonnaux, C., Johnson, R.S., Cramer, T., Sobolewski, A., Condliffe, A.M., Cowburn, A.S., Johnson, N., Chilvers, E.R., 2005. Hypoxia-induced neutrophil survival is mediated by HIF-1α-dependent NF-κB activity. J. Exp. Med. 201, 105-115.

Albina, J.E., Mastrofrancesco, B., Vessella, J.A., Louis, C.A., Henry, W.L., Reichner, J.S., 2001. HIF-1 expression in healing wounds: HIF-1α induction in primary inflammatory cells by TNF-α. Am. J. Physiol. Cell Physiol. 281, C1971-C1977

Blouin, C.C., Pagé, E.L., Soucy, G.M., Richard, D.E., 2004. Hypoxic gene activation by lipopolysaccharide in macrophages: Implication of hypoxia-inducible factor 1α. Blood. 103, 1124-1130.

Fitzpatrick, S.F., Tambuwala, M.M., Bruning, U., Schaible, B., Scholz, C.C., Byrne, A., O′Connor, A., Gallagher, W.M., Lenihan, C.R., Garvey, J.F., Howell, K., Fallon, P.G., Cummins, E.P., Taylor, C.T., 2011. An Intact Canonical NF-κB Pathway Is Required for Inflammatory Gene Expression in Response to Hypoxia. J. Immunol. 186, 1091-1096.

Scholz, C.C., Cavadas, M.A.S., Tambuwala, M.M., Hams, E., Rodríguez, J., Von Kriegsheim, A., Cotter, P., Bruning, U., Fallon, P.G., Cheong, A., Cummins, E.P., Taylor, C.T., 2013. Regulation of IL-1β-induced NF-κB by hydroxylases links key hypoxic and inflammatory signaling pathways. Proc. Natl. Acad. Sci. U. S. A. 110, 18490-18495.

Robinson, A., Keely, S., Karhausen, J., Gerich, M.E., Furuta, G.T., Colgan, S.P., 2008. Mucosal Protection by Hypoxia-Inducible Factor Prolyl Hydroxylase Inhibition. Gastroenterology 134, 145-155.

Tambuwala, M.M., Cummins, E.P., Lenihan, C.R., Kiss, J., Stauch, M., Scholz, C.C., Fraisl, P., Lasitschka, F., Mollenhauer, M., Saunders, S.P., Maxwell, P.H., Carmeliet, P., Fallon, P.G., Schneider, M., Taylor, C.T., 2010. Loss of prolyl hydroxylase-1 protects against colitis through reduced epithelial cell apoptosis and increased barrier function. Gastroenterology 139, 2093-2101.

Manresa, M.C., Smith, L., Casals-Diaz, L., Fagundes, R.R., Brown, E., Radhakrishnan, P., Murphy, S.J., Crifo, B., Strowitzki, M.J., Halligan, D.N., van den Bogaard, E.H., Niehues, H., Schneider, M., Taylor, C.T., Steinhoff, M., 2019. Pharmacologic inhibition of hypoxia-inducible factor (HIF)-hydroxylases ameliorates allergic contact dermatitis. Allergy Eur. J. Allergy Clin. Immunol. 74, 753-766.

Jain, M., Joharapurkar, A., Patel, V., Kshirsagar, S., Sutariya, B., Patel, M., Patel, H., Patel, P.R., 2019. Pharmacological inhibition of prolyl hydroxylase protects against inflammation-induced anemia via efficient erythropoiesis and hepcidin downregulation. Eur. J. Pharmacol. 843, 113-120.

Joharapurkar, A.A., Pandya, V.B., Patel, V.J., Desai, R.C., Jain, M.R., 2018. Prolyl Hydroxylase Inhibitors: A Breakthrough in the Therapy of Anemia Associated with Chronic Diseases. J. Med. Chem. 61, 6964-6982

1. Prolyl hydroxylase inhibitors for the treatment of Psoriasis and skin inflammatory diseases.

2. Prolyl hydroxylase inhibitors as claimed in claim 1 wherein Prolyl hydroxylase inhibitors are selected from Molidustat, Vadadustat and Daprodustat and compound of formula (Ia).

3. Pharmaceutical composition comprising prolyl hydroxylase inhibitors or pharmaceutically acceptable salts thereof and suitable pharmaceutically acceptable excipients for the treatment of Psoriasis and skin inflammatory diseases.

4. Pharmaceutical composition as claimed in claim 3 wherein prolyl hydroxylase inhibitors are selected from Molidustat, Vadadustat and Daprodustat and compound of formula (Ia).

5. Pharmaceutical composition comprising compound of formula (Ia) or its pharmaceutically acceptable salts and suitable pharmaceutically acceptable excipients for the treatment of Psoriasis and skin inflammatory diseases wherein compound of formula (Ia) is 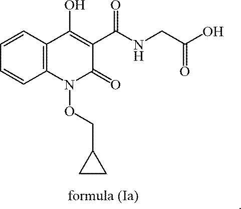
.

6. Pharmaceutical composition as claimed in claim 5 wherein pharmaceutically acceptable salts are selected from calcium, sodium, potassium, lithium, barium, strontium, magnesium, cesium, copper, cobalt, iron, manganese, lead, aluminium, cadmium, silver, zinc, ammonium, methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (-)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane1-6-diamine, 2-(2-aminoethoxy)ethanamine, N-methylmorpholine, and N-ethylmorpholine.

7. Pharmaceutical composition as claimed in claim 5 wherein pharmaceutically acceptable excipients are selected from diluent, binders, disintegrating agents, lubricating agents, glidant agent, coating redimix, gelling agent, humectant, chelating agents, permeation enhancers, preservatives, antioxidants, solubilizing agents, acidifying/alkalizing agent, Emollient and Emulsifying agents.

8. Pharmaceutical composition as claimed in claim 7 wherein diluents are selected from maltitol, lactitol, microcrystalline cellulose, magnesium or calcium or sodium carbonate, lactose, lactose monohydrate, di-calcium phosphate, compressible sugars, di-basic calcium phosphate dihydrate, mannitol lactose anyhydrous, magnesium oxide, maltodextrin, maltose, pullulan, sodium alginate, sodium bicarbonate, calcium silicate, calcium sulphate, cell and tribasic calcium phosphate or suitable combinations thereof.

9. Pharmaceutical composition as claimed in claim 7 wherein disintegrating agents are selected from maize starch, sodium starch glycolate, croscarmellose sodium, crospovidone, microcrystalline cellulose, modified com starch, sodium carboxymethyl starch, povidone, pregelatinized starch, agar, carboxymethyl cellulose calcium or sodium, colloidal silicon dioxide, chitosan, docusate sodium, hydroxyl propyl cellulose, magnesium aluminium silicate, maltose, methyl cellulose, polacrilin potassium, and alginic acid or suitable combinations thereof.

10. Pharmaceutical composition as claimed in 7 wherein lubricating agents are selected from lubricants include magnesium stearate, stearic acid, silica, fats, zinc or sucrose or sodium or calcium stearate, castor oil, hydrogenated castor oil, . Polyethylene glycol and its derivatives, sodium stearyl fumarate, talc, or fatty acids including lauric acid, oleic acid, glyceryl behenate, glyceryl monostearate, and C₁-C₁₀ fatty acid or suitable combinations thereof.

11. Pharmaceutical composition as claimed in claim 7 wherein humectant is selected from glycerin, propylene glycol, Polyethylene glycol, sorbitol solution, 1,2,6 -hexanetriol and suitable mixtures thereof.

12. Pharmaceutical composition as claimed in claim 7 wherein gelling agents are selected from carbomer, Methyl cellulose, sodium carboxy methyl clellulose, Carrageenan, colloidal silicon dioxide, Guar gum, hydroxypropyl methyl cellulose, hydroxyl ethyl cellulose, hydroxy propyl cellulose, Gelatin, polyethyene oxide, alginic acid, sodium alginate, fumed silica, polyvinylpyrrolidone, polyvinyl alcohol and suitable mixtures thereof.

13. Pharmaceutical composition as claimed in claim 7 wherein emollient are selected from carnauba wax, cetyl alcohol, cetyl ester wax, hydrous lanolin, lanolin, lanolin alcohols, paraffin, petrolatum. polyethylene glycol, stearic acid, stearyl alcohol, white wax, yellow wax, liquid paraffin, liquid petrolatum, jojoba oil, sesame oil, rapeseed oil, purcellin oil, 2-ethylhexyl palmitate, 2-octyldodecyl stearate, 2-octyldodecyl erucate, isostearyl isostearate, 2-octyldodecyl benzoate, triglycerides of caprylic/capric acids, octyldodecanol, isohexadecane, capmul MCM and suitable mixtures thereof.

14. Pharmaceutical composition as claimed in claim 7 wherein alkalizing agents are selected from trimethylamine, triethylamine, pyridine, picoline, ethanolamine, diethanolamine, triethanolamine, meglumine, dicyclohexylamine, N,N′-dibenzylethylenediamine, arginine, lysine, ornithine, sodium bicarbonates, sodium hydroxide, potassium hydroxide and suitable mixtures thereof.

15. Pharmaceutical composition as claimed in claim 7 wherein binders are selected from hypromellose 3 Cps, carbomers selected from carbopol, gellan, gum Arabic, hydrogenated vegetable oil, polymethacrylates selected from Eudragit, xanthan, lactose and Zein combinations thereof; glidant agents are selected from colloidal silica, calcium silicate, magnesium silicate, silicon hydrogel, cornstarch, talc, combinations thereof.

16. Pharmaceutical composition as claimed in claim 7 wherein chelating agents are selected from EDTA, disodium EDTA, trisodium EDTA, EGTA, disodium EGTA, trisodium EGTA, citric acid, phosphoric acid, succinic acid, and suitable mixtures thereof; permeation enhancers are selected from polyethylene glycol, polyethylene glycol monolaurate, butanediol, dimethylsulfoxide, decylmethylsulfoxide, diethylene glycol monoethyl ether, lauric acid, oleic acid, valeric acid, isopropyl myristate, isopropyl palmitate, methyl propionate, and ethyl oleate, urea, dimethyl acetamide, dimethylformamide 2- pyrrolidone, ethanolamine, methyl-2 -pyrrolidone, diethanolamine, triethanolamine, terpenes, alkanones, salicylic acid, citric acid, succinic acid and suitable mixtures thereof; preservatives are selected from Methyl paraben, Propyl paraben, benzoic acid, imidurea, sorbic acid, potassium sorbate, benzalkonium chloride, phenyl mercuric acetate, chlorobutanol, phenoxyethanol, benzyl alcohol, chlorocresol, metacresol, cetrimonium chloride, benzethonium chloride, sodium edetate, boric acid, phenol and suitable mixtures thereof; antioxidants are selected from ascorbic acid, glutathione, lipoic acid, uric acid, carotenes, α-tocopherol, ubiquinol, butylated hydroxyanisole, butylated hydroxytoluene, sodium benzoate, sodium thiosulphate, sodium metabisulphite, propyl gallate, and tertiary-butylhydroquinone, Idebenone, Lycopene and suitable mixtures thereof; and solubilizing agents are selected from Dimethyl malonate, diethyl succinate, diethyl glutarate, diethyl adipate, dipropyl adipate, dibutyl sebacate, diisopropyl sebacate, diethyl pimelate, diethyl suberate, diethyl azelate, dibutyl adipate, dibutyl sebacate, methyl ethyl succinate, diethyl ethyl-isopropylmalonate, diethyl isosuccinate, benzyl alcohol, benzyl benzoate, cyclodextrin, glycerine monostearate, lecithin, butylene glycol, dibutyl phthalate, diethyl phthalate, dimethyl ether, diethyl ether, ethyl acetate, ethyl lactate, ethyl oleate, glycofurol, isopropyl alcohol, triacetin, triethanolamine, hexylene glycol, dimethyl sulfoxide (DMSO) and/or dimethyl isosorbide, propylene glycol, glycerin, Diethylene glycol monoethyl ether, dimethyl acetamide, polyethylene glycol, polysorbate 80, 60 & 20, purified water, ethanol and suitable mixture thereof.

17. Pharmaceutical composition as claimed in claim 3 wherein effective amount of compound of formula (Ia) is ranging from 1% to 10%w/w.

18. Pharmaceutical composition as claimed in claim 3 is administered orally, intravenously, parentally or topically.

19. Pharmaceutical composition as claimed in claim 5 further comprising additional therapeutic agents.

20. Pharmaceutical composition as claimed in claim 19 wherein additional therapeutic agents are selected from class of a PDE4 inhibitor, methotrexate or derivatives thereof, cyclosporine or derivatives thereof, vitamins D, E and A or derivatives thereof, glucocorticoid or derivatives thereof, anti-inflammatory agents, immunosuppressive agents, antihistamines, monoclonal antibodies and fusion proteins, cytokines and mediators, antibiotic or derivatives thereof, antifungal agents or derivatives thereof, hormones, peroxisome proliferator-activated receptor gamma activators or derivatives thereof, anti-viral agents or derivatives thereof, vegetable and/or animal extracts, phytochemicals or derivatives thereof, zinc pyrithione and/or other essential minerals, phototherapy and cell hyper proliferation modulators.

21. Compound of formula (Ia) or its pharmaceutically acceptable salts for the treatment of Psoriasis and skin inflammatory diseases wherein compound of formula (Ia) is 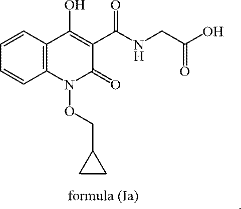
.

22. Compound of formula (Ia) or its pharmaceutically acceptable salts as claimed in claim 12 wherein pharmaceutically acceptable salts are selected from calcium, sodium, potassium, lithium, barium, strontium, magnesium, cesium, copper, cobalt, iron, manganese, lead, aluminium, cadmium, silver, zinc, ammonium, methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenyl ethylamine, (S)-4-methoxyphenylethylamine, (N)-4-chlorophenylethylamine, (S)-4-methylphenylethylamine, cinchonine, cinchonidine, (-)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane1-6-diamine, 2-(2-aminoethoxy)ethanamine, N-methylmorpholine, and N-ethylmorpholine.

23. Method of treating Psoriasis and skin inflammatory diseases using compound of formula (Ia) or its pharmaceutically acceptable salts wherein compound of formula (Ia) is 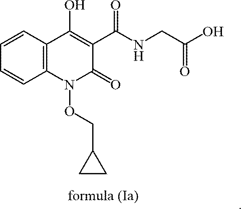
.
